(19) United States
(12) Patent Application Publication
(10) Pub. No.: US 2016/0263365 A1
(43) Pub. Date: Sep. 15, 2016

(54) Rotary Tattoo Machine with Improved Dampening

(71) Applicant: Todd William Smith, Fort Myers, FL (US)

(72) Inventor: Todd William Smith, Fort Myers, FL (US)

(21) Appl. No.: 15/065,646

(22) Filed: Mar. 9, 2016

Related U.S. Application Data
(60) Provisional application No. 62/130,342, filed on Mar. 9, 2015.

(51) Int. Cl. A61M 37/00

(52) U.S. Cl. CPC A61M 37/0076

(57) ABSTRACT

A rotary tattoo machine utilizes a spring-mass system to provide a dampening effect during operation. The rotary tattoo machine includes a tattoo machine housing, a reciprocating slider, and a dampening slider. A first channel traverses into the tattoo machine housing and receives the reciprocating slider. The reciprocating slider includes a first main body, a plurality of first bearings, a second channel, and a removable endcap. Each of the first bearings is rotatably connected along the first main body. Additionally, the first bearings are slidably engaged with the first channel. The second channel traverses into the first main body, parallel to the first channel. The dampening slider absorbs reactionary forces and includes a second main body, a plurality of second bearings, and a first spring. Each of the second bearings is rotatably connected along the second main body. Additionally, the second bearings are slidably engaged with the second channel.

The current application claims a priority to the U.S. Provisional Patent application Ser. No. 62/130,342 filed on Mar. 9, 2015.

### FIELD OF THE INVENTION

The present invention relates generally to tattoo machines. More particularly, the present invention is a rotary tattoo machine with improved dampening which partially or fully absorbs the force resulting when the needle impacts the skin.

### BACKGROUND OF THE INVENTION

Tattoo machines have been in use for many years. A tattoo machine typically has a reciprocating needle that moves up and down within a tubular structure, carrying ink into the skin of an individual in the process. The reciprocating needle typically punctures the skin at a high rate. The needles are installed in the machine and dipped in ink, which is sucked up through the machine's tube system. Then, the tattoo machine induces an up-and-down motion of the needle to puncture the top layer of the skin and drive insoluble particles of ink into the dermal layer of skin.

The two most common tattoo machines currently in use are coil tattoo machines and rotary tattoo machines. The latter uses the rotation of an electric motor to drive the needles. In some rotary machines, the needle is eccentrically connected to the output shaft of the motor by a cam wheel, which allows the needle to move in a reciprocating motion. Others function similarly as a crank mechanism, where the circular motion of the motor output shaft is converted into perfect linear reciprocation of the needle by an interconnecting rod. Rotary tattoo machines can offer several advantages to the coil machines in that a rotary tattoo machine is typically lighter, significantly quieter, and can be used as either a liner or a shader. However, rotary tattoo machines typically do not have much in the way of dampening the vibrations of the needles; the needles do not “give” as they puncture the skin of the individual being tattooed. This can result in accelerated wear and tear of the machine as well as discomfort for the tattoo artist and decreased accuracy. The present invention is a rotary-type tattoo machine with improved dampening to absorb the force resulting when an ink-carrying needle bar connected thereto punctures the skin.

### BRIEF DESCRIPTION OF THE DRAWINGS

FIG. 1 is a perspective view of the present invention.

FIG. 2 is an exploded perspective view of the present invention.

FIG. 3 is an exploded perspective view of the reciprocating slider, the dampening slider, the dampening adjustment assembly, and the second spring.

FIG. 4 is a front view of the reciprocating slider, the dampening adjustment assembly, and the first spring depicting the staggered pattern which is followed by the plurality of first bearings.

FIG. 5 is a front view of the dampening slider depicting the staggered pattern which is followed by the plurality of second bearings follow.

FIG. 6 is a perspective view of an alternative embodiment of the present invention.

FIG. 7 is a side-view of the alternative embodiment of the present invention.

### DETAIL DESCRIPTIONS OF THE INVENTION

All illustrations of the drawings are for the purpose of describing selected versions of the present invention and are not intended to limit the scope of the present invention.

The present invention generally relates to tattoo machine designs. More specifically, the present invention is a rotary-type tattoo machine which utilizes a spring-mass system to dampen/decrease a reactionary force applied to the tattoo machine in response to the needle impacting/penetrating the skin. Reducing the reactionary force increases the accuracy of the tattoo artist while simultaneously decreasing wear and tear on the tattoo machine. Additionally, this increases the comfort level for the tattoo artist and the individual receiving the tattoo.

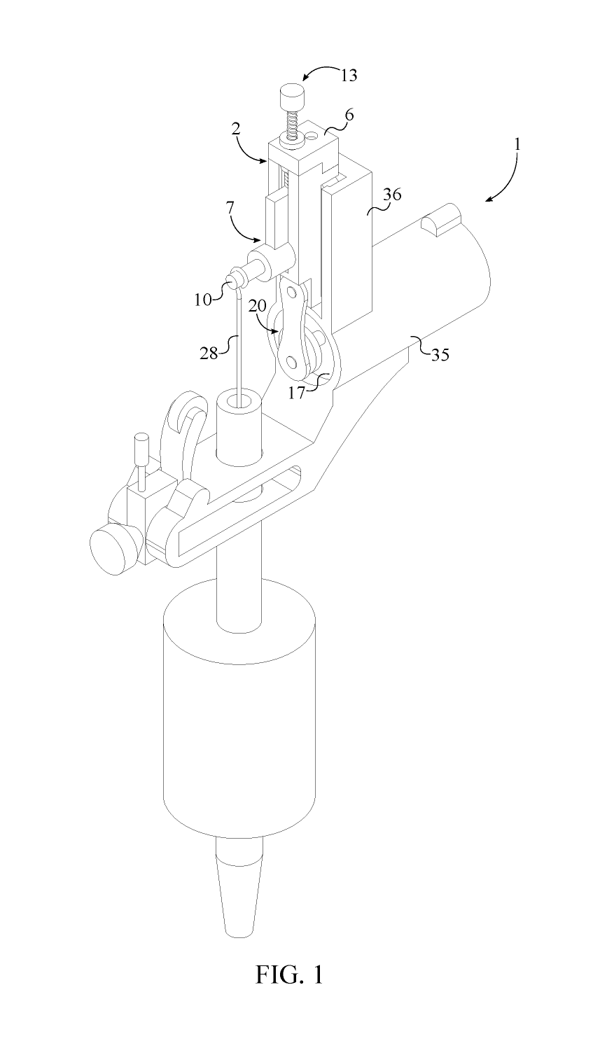

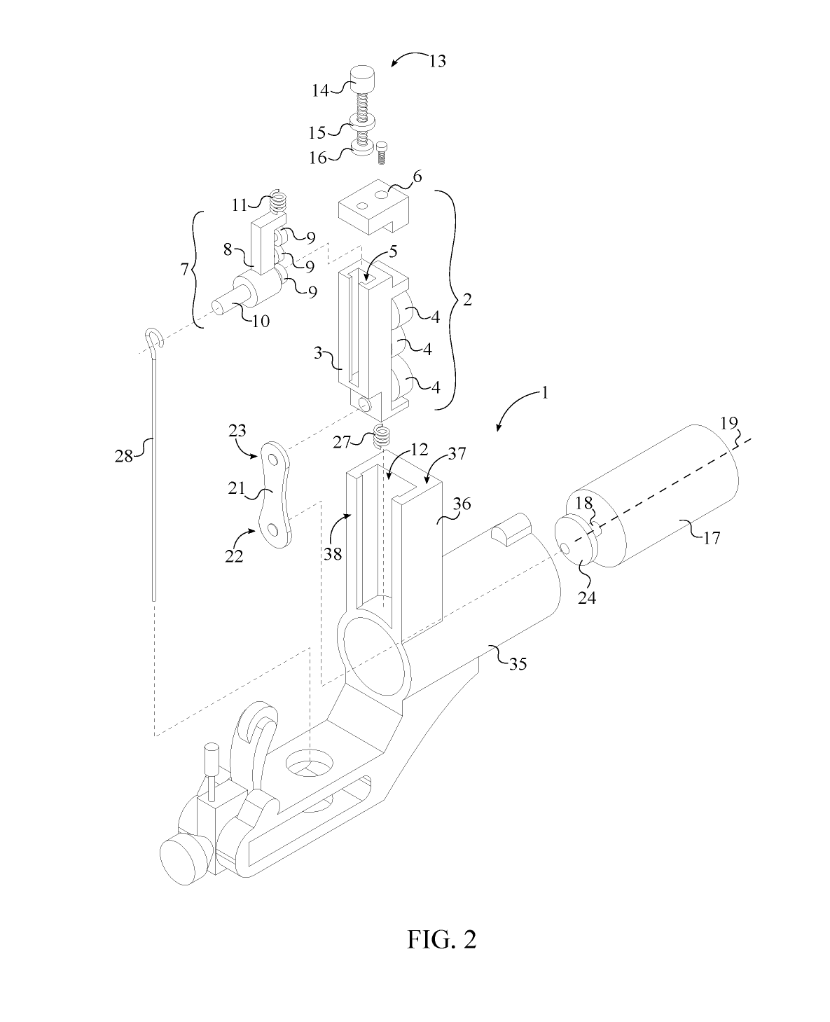

Referring to FIG. 1 and FIG. 2, the present invention comprises a tattoo machine housing 1, a reciprocating slider 2, a dampening slider 7, a motor 17, and a rotary-to-linear mechanical linkage 20. The tattoo machine housing 1 retains and supports the constituents of the present invention. The tattoo machine housing 1 comprises a motor-receiving tube 35 and a tower housing 36. The tower housing 36 is adjacently and perpendicularly connected to the motor-receiving tube 35, similar to traditional tattoo machine designs. The reciprocating slider 2 in conjunction with the motor 17 and the rotary-to-linear mechanical linkage 20 generate the reciprocating linear motion necessary for the operation of the present invention. The dampening slider 7 transfers said motion to a needle bar 28 and reduces/absorbs the reactionary forces. The needle bar 28 includes an at least one needle that is used to drive ink into the skin.

Referring to FIG. 2, the reciprocating slider 2 is slidably engaged to the tattoo machine housing 1 through a first channel 12. The first channel 12 traverses into the tattoo machine housing 1 and is shaped to receive a portion of the reciprocating slider 2. More specifically, the first channel 12 traverses into the tower housing 36 from a top surface 37 of the tower housing 36, intersecting a front surface 38 of the tower housing 36 as seen in FIG. 2; this configuration creates two lateral flanges that prevent the reciprocating slider 2 from disconnecting from the tower housing 36. The reciprocating slider 2 comprises a first main body 3, a plurality of first bearings 4, a second channel 5, and a removable endcap 6. The plurality of first bearings 4 allows the reciprocating slider 2 to linearly translate within the first channel 12 with minimal energy losses due and with minimum lateral shifting. This decreasing the overall vibration of the present invention during operation. Each of the plurality of first bearings 4 is rotatably connected along the first main body 3. In addition, the plurality of first bearings 4 is slidably engaged with the first channel 12 through the lateral flanges. This configuration allows the plurality of first bearings 4 to interact with the lateral walls of the first channel 12 in a rolling engagement. The second channel 5 traverses into the first main body 3, oriented parallel to the first channel 12, and houses/receives the dampening slider 7. The second channel 5 and the plurality of first bearings 4 are oppositely positioned of each other, across the first main body 3.

The removable endcap 6 closes off the second channel 5 and retains the dampening slider 7 within the second channel 5. More specifically, the removable endcap 6 is attached to the first main body 3, adjacent to an inlet 39 of the second channel 5. The removable endcap 6 is preferably attached to the first main body 3 by a fastening bolt in order to allow for easy disassembly and assembly, essential for cleaning and modularity purposes. As mentioned above, the dampening slider 7 transfers the reciprocating motion of the reciprocating slider 2 to the needle bar 28. In addition, the dampening slider 7 acts as a dampening mass-spring system in order to decrease and dampen the reactionary force resulting when the needle bar 28 punctures the skin. In other words, the dampening slider 7 provides the needle bar 28 with a certain amount of “give” when engaging/impacting the skin. This is achieved by connecting the dampening slider 7 directly to the needle bar 28, the source of the reactionary force.

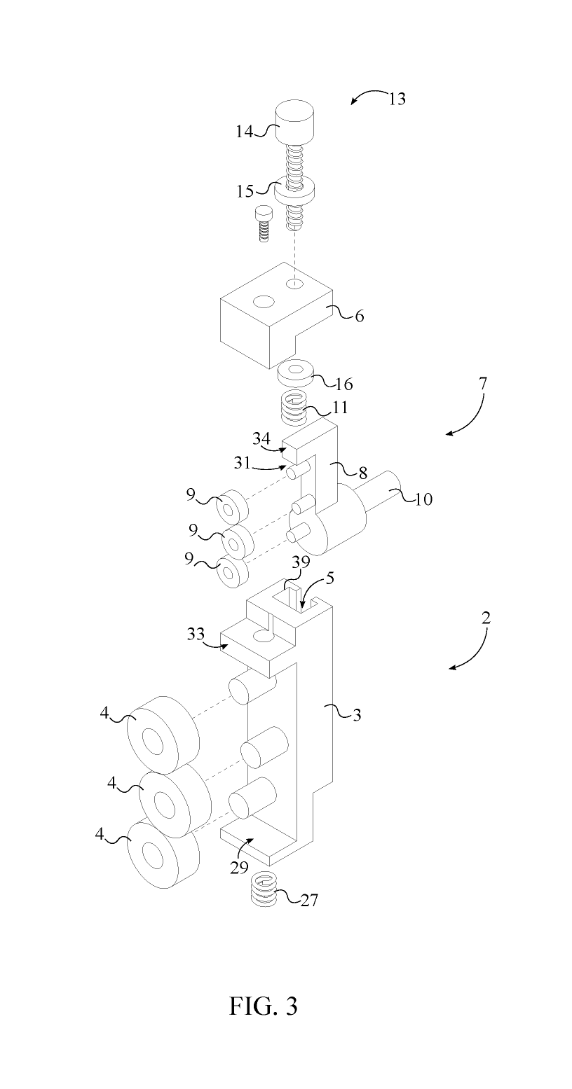

Referring to FIG. 3, the dampening slider 7 comprises a second main body 8, a plurality of second bearings 9, a needle-connecting rod 10, and a first spring 11. The second main body 8 acts as the mass in the dampening mass-spring system. Similar to the reciprocating slider 2, the plurality of second bearings 9 allows the dampening slider 7 to linearly translate within the second channel 5 with minimal energy losses and with minimum lateral shifting, therefore decreasing the overall vibration of the present invention. More specifically, each of the plurality of second bearings 9 is rotatably connected along the second main body 8. The plurality of second bearings 9 is additionally slidably engaged with the second channel 5. This allows the plurality of second bearings 9 to interact with the lateral walls of the second channel 5 in a rolling engagement. The first spring 11 acts as the elastic element in the dampening mass-spring system and is positioned in between the removable endcap 6 and the second main body 8. The first spring 11 absorbs a portion of the reactionary force applied to the needle bar 28 and prevents harsh impacts between the second main body 8 and the removable endcap 6 during operation of the present invention. The needle-connecting rod 10 attaches the dampening slider 7 to the needle bar 28 and is therefore perpendicularly and adjacently connected to the second main body 8, opposite the plurality of second bearings 9. Referring to FIG. 1, the needle bar 28 is positioned adjacent to the needle-connecting rod 10, oriented parallel to the first channel 12, and is perpendicularly attached to the needle-connecting rod 10.

The motor 17 converts electrical energy into mechanical energy, in the form of rotational motion to be more specific. The motor 17 is preferably a conventional direct current (DC) electric motor with an output shaft 18 extending from an end thereof. The motor 17 is mounted within the motor-receiving tube 35 with the output shaft 18 of the motor 17 positioned adjacent to the first channel 12. This positions the output shaft 18 near the reciprocating slider 2. The output shaft 18 is mechanically coupled to the first main body 3 by the rotary-to-linear mechanical linkage 20. The rotary-to-linear mechanical linkage 20 takes the rotational motion of the motor 17 and applies it to the reciprocating slider 2. In response to this motion, the reciprocating slider 2 translates in a linearly-oscillating fashion as the reciprocating slider 2 is restricted only to linear translation by the first channel 12.

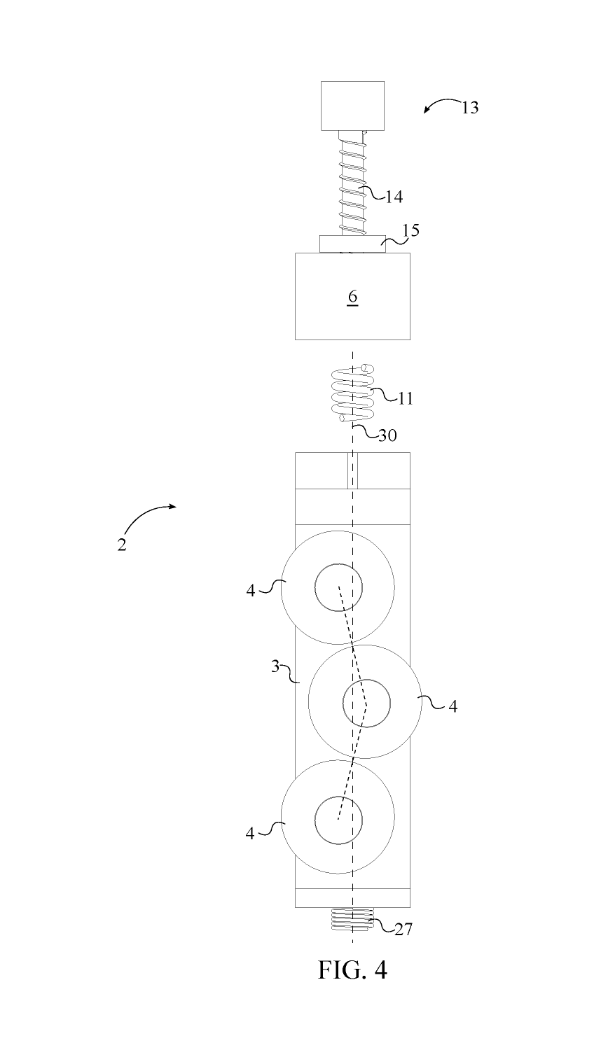

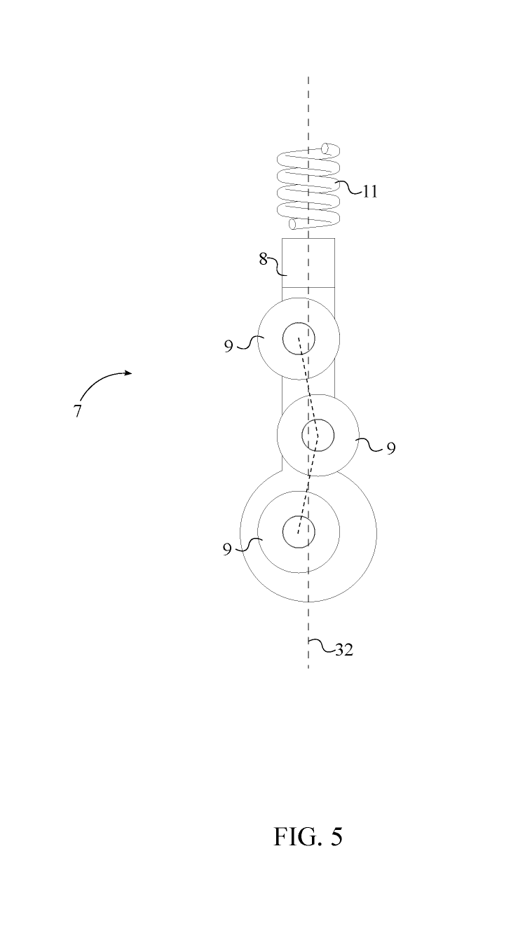

Referring to FIG. 4 and FIG. 5, in the preferred embodiment, the present invention minimizes energy lost during the conversion of rotational motion into linear reciprocation motion through the plurality of first bearings 4 and the plurality of second bearings 9. More specifically, the reciprocating slider 2 further comprises a first bearing-receiving cavity 29. The first bearing-receiving cavity 29 traverses into the first main body 3 from a rear surface 33 of the first main body 3. The plurality of first bearings 4 is positioned within the first bearing-receiving cavity 29. In particular, the plurality of first bearings 4 is distributed about a centerline 30 of the first main body 3 along a staggered pattern; alternating between left-skewed and right-skewed positions relative to the centerline 30 of the first main body 3 as seen in FIG. 4. This configuration prevents lateral shifting during the reciprocating motion. Additionally, this configuration allows a first set of bearings within the plurality of first bearings 4 to engage a first lateral wall of the first channel 12 while a second set of bearings within the plurality of first bearings 4 engages a second lateral wall of the first channel 12, resulting in a smooth linear transition as each set of bearings spin in opposite directions.

Referring FIG. 3, the dampening slider 7 further comprises a second bearing-receiving cavity 31. The second bearing-receiving cavity 31 traverses into the second main body 8 from a rear surface 34 of the second main body 8. The plurality of second bearings 9 is positioned within the second bearing-receiving cavity 31. In particular, the plurality of second bearings 9 is distributed about a centerline 32 of the second main body 8 along a staggered pattern; alternating between left-skewed and right-skewed positions relative to the centerline 32 of the second main body 8 as seen in FIG. 5. This configuration prevents lateral shifting during reciprocation. Additionally, this configuration allows a set of bearings within the plurality of second bearings 9 to engage a first lateral wall of the second channel 5 while a second set of bearings within the plurality of second bearings 9 engages a second lateral wall of the second channel 5, resulting in a smooth linear transition as each set of bearings spin in opposite directions.

Referring to FIG. 3, the present invention further comprises a second spring 27. The second spring 27 is positioned within the first channel 12, between the tower housing 36 and the first main body 3. The second spring 27 provides a cushioning that prevents harsh impacts between the reciprocating slider 2 and the bottom end of the first channel 12. The second spring 27 is orientated parallel to the first channel 12. Additionally, the second spring 27 is externally connected to the first main body 3, opposite the removable endcap 6. The second spring 27 is a compression spring which biases the reciprocating slider 2 towards an open distal end of the first channel 12. This positions the reciprocating slider 2 at a highest position, or on the upper stroke of reciprocating motion, when the motor 17 is de-energized. In this manner, the needle bar 28 is always retracted away from the skin when the present invention is turned off.

Referring to FIG. 2, the present invention further comprises a dampening adjustment assembly 13 which allows the user to restrict the range of movement of the dampening slider 7, thus varying the dampening effect produced by the dampening slider 7. The dampening adjustment assembly 13 comprises a thumbscrew 14, a first nut 15, and a second nut 16. The first nut 15 is positioned adjacent to the removable endcap 6, opposite the second channel 5, and acts as a locking nut in order to prevent vibrations from loosening the thumbscrew 14 as the dampening slider 7 reciprocates. The second nut 16 is positioned within the second channel 5, parallel to the first nut 15. The second nut 16 provides a surface for the first spring 11 to press against. The thumbscrew 14 is positioned collinear with the second channel 5 and traverses through the first nut 15, the removable endcap 6, and the second nut 16. More specifically, the thumbscrew 14 traverses through the second nut 16 such that a portion of the thumbscrew 14 extends past the second nut 16. This provides an anchor point for the first spring 11. The first spring 11 is positioned concentric with the thumbscrew 14, within the second channel 5. More specifically, the first spring 11 is positioned in between the first nut 15 and the second main body 8, pressing against both components. In the preferred embodiment, the thumbscrew 14 is of suitable length such that it can be tightened until the dampening slider 7 is completely restrained against a bottom end of the second channel 5. As a result, the user can adjust the amount of “give” in the needle bar 28, i.e., the amount of force absorbed when the needle bar 28 punctures the skin.

Referring to FIG. 2, in one embodiment of the present invention, the rotary-to-linear mechanical linkage 20 is a slider-crank mechanism. In this embodiment, the rotary-to-linear mechanical linkage 20 comprises a connecting arm 21 and an interconnecting disk 24. The interconnecting disk 24 is concentrically connected to the output shaft 18. Through the interconnecting disk 24 the connecting arm 21 connects the output shaft 18 to the reciprocating slider 2. More specifically, a first end 22 of the connecting arm 21 is pivotably connected to the interconnecting disk 24, and the first end 22 is radially offset from the main axis 19 of the output shaft 18. A second end 23 of the connecting arm 21 is adjacently positioned and pivotably connected to the first main body 3, opposite the plurality of first bearings 4. As the output shaft 18 rotates the interconnecting disk 24, the first end 22 of the connecting arm 21 travels in a circular path about the main axis 19 of the output shaft 18, while the second end 22 of the connecting arm 21 is constrained by the reciprocating slider 2 to move in a linear reciprocating motion. This reciprocating motion then moves the dampening slider 7 and therefore moves the needle-connecting rod 10 and the needle bar 28.

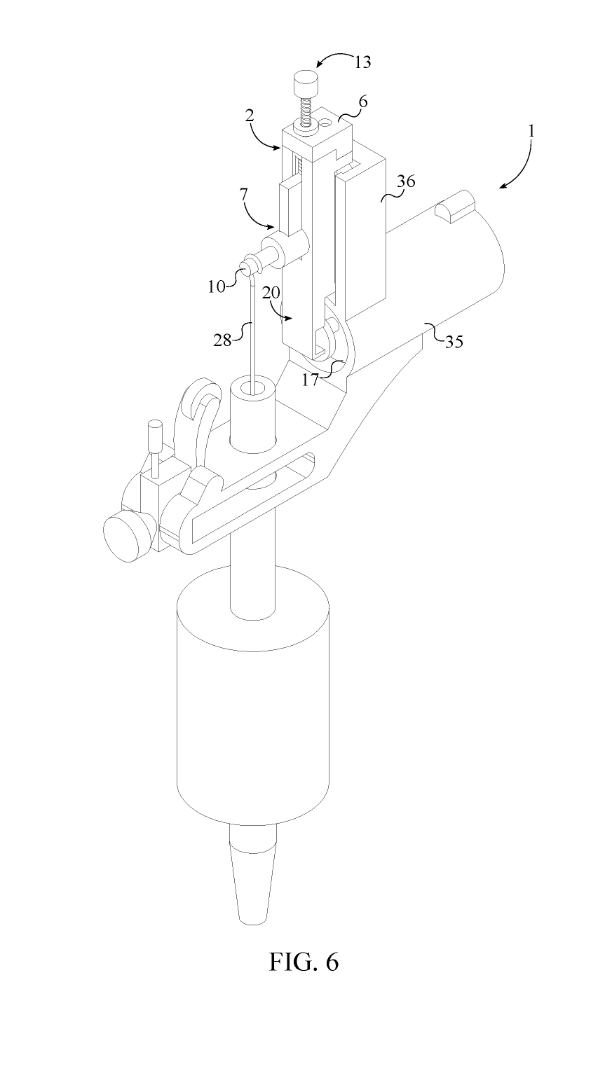

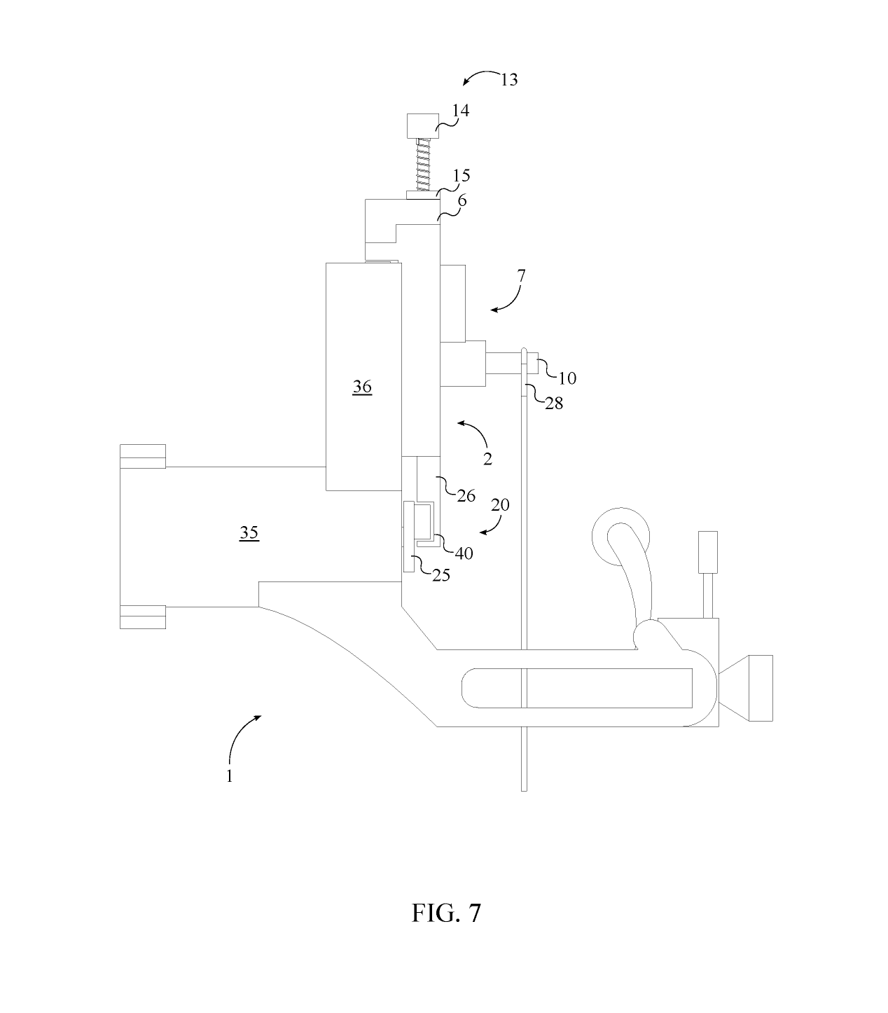

Referring to FIG. 6 and FIG. 7, in another embodiment of the present invention, the rotary-to-linear mechanical linkage 20 utilizes a cam-type mechanism. In this embodiment, the rotary-to-linear mechanical linkage 20 comprises a cam 25, a translating arm 26, and a cam-receiving cavity. The cam 25 is axially connected to the output shaft 18. The translating arm 26 is adjacently connected to the first main body 3, opposite the removable endcap 6. The cam-receiving cavity 40 traverses into the translating arm 26 and is shaped to receive the cam 25. The cam 25 is perimetrically engaged within the cam-receiving cavity 40 as seen in FIG. 40. When the output shaft 18 rotates the cam 25, the cam 25 will spin inside the cam-receiving cavity 40, engaging the upper and lower walls of the cam-receiving cavity 40. This will raise and lower the translating arm 26 and, therefore moving the adjoined reciprocating slider 2.

The tattoo machine housing 1 may further comprise a needle bar retainer assembly, which includes a retainer bearing that exerts pressure on the needle bearing in order to keep the eyelet of the needle bar 28 fixedly mounted to the needle-connecting rod 10. A retainer screw is used to tighten or loosen the retainer bearing. When the retainer is screw is loosened, the needle bar 28 can be removed from the tattoo machine housing 1. Such a needle bar 28 retainer assembly configuration can be similar to the needle bar 28 retainers employed in conventional tattoo machines.

Although the invention has been explained in relation to its preferred embodiment, it is to be understood that many other possible modifications and variations can be made without departing from the spirit and scope of the invention as hereinafter claimed.

What is claimed is:

1. A rotary tattoo machine with improved dampening comprises:
a tattoo machine housing;
a reciprocating slider comprises a first main body, a plurality of first bearings, a second channel, and a removable endcap;
a dampening slider comprises a second main body, a plurality of second bearings, a needle-connecting rod, and a first spring;
a first channel traversing into the tattoo machine housing;
each of the plurality of first bearings being rotatably connected along the first main body;
the plurality of first bearings being slidably engaged with the first channel;
each of the plurality of second bearings being rotatably connected along the second main body;
the second channel traversing into the first main body, oriented parallel to the first channel;
the second channel and the plurality of first bearings being oppositely positioned of each other across the first main body;
the plurality of second bearings being slidably engaged with the second channel;
the needle-connecting rod being perpendicularly and adjacently connected to the second main body, opposite the plurality of second bearings; and
the removable endcap being attached to the first main body, adjacent to an inlet of the second channel.

2. The rotary tattoo machine with improved dampening as claimed in claim 1 comprises:
a dampening adjustment assembly;
the dampening adjustment assembly comprises a thumbscrew, a first nut, and a second nut;
the thumbscrew being positioned collinear with second channel;
the first nut being positioned adjacent to the removable endcap, opposite the second channel;
the second nut being positioned within the second channel, parallel to the first nut;
the thumbscrew traversing through the first nut, the removable endcap, and the second nut;
the first spring being positioned within the second channel, concentric with the thumbscrew; and
the first spring being positioned in between the first nut and the second main body.

3. The rotary tattoo machine with improved dampening as claimed in claim 1 comprises:
the tattoo machine housing comprises a motor-receiving tube and a tower housing;
the tower housing being adjacently and perpendicularly connected to the motor-receiving tube;
the first channel traversing into the tower housing from a top surface of the tower housing; and
the first channel intersecting a front surface of the tower housing.

4. The rotary tattoo machine with improved dampening as claimed in claim 1 comprises:
a motor;
a rotary-to-linear mechanical linkage;
the motor being mounted within a motor-receiving tube of the tattoo machine housing;
an output shaft of the motor being positioned adjacent to the first channel; and
the output shaft being mechanically linked to the first main body by the rotary-to-linear mechanical linkage.

5. The rotary tattoo machine with improved dampening as claimed in claim 1 comprises:
a rotary-to-linear mechanical linkage;
a motor;
the rotary-to-linear mechanical linkage comprises a connecting arm and an interconnecting disk;
the interconnecting disk being concentrically connected to an output shaft of the motor;
a first end of the connecting arm being pivotably connected to the interconnecting disk;
the first end being positioned radially offset from a main axis of the output shaft;
a second end of the connecting arm being adjacently positioned to the first main body, opposite the plurality of first bearings; and
the second end being pivotably connected to the first main body.

6. The rotary tattoo machine with improved dampening as claimed in claim 1 comprises:
a rotary-to-linear mechanical linkage;
a motor;
the rotary-to-linear mechanical linkage comprises a cam, a translating arm, and a cam-receiving cavity;
the cam being axially connected to an output shaft of the motor;
the translating arm being adjacently connected to the first main body, opposite the removable endcap;
the cam-receiving cavity traversing into the translating arm; and
the cam being perimetrically engaged within the cam-receiving cavity.

7. The rotary tattoo machine with improved dampening as claimed in claim 1 comprises:
a second spring;
the second spring being oriented parallel to the first channel;
the second spring being externally connected to the first main body, opposite the removable endcap; and
the second spring being positioned within the first channel.

8. The rotary tattoo machine with improved dampening as claimed in claim 1 comprises:
a needle bar;
the needle bar being positioned adjacent to the needle-connecting rod;
the needle bar being oriented parallel to the first channel; and
the needle bar being perpendicularly attached to the needle-connecting rod.

9. The rotary tattoo machine with improved dampening as claimed in claim 1 comprises:
the reciprocating slider further comprises a first bearing-receiving cavity;
the first bearing-receiving cavity traversing into the first main body from a rear surface of the first main body;
the plurality of first bearings being positioned within the first bearing-receiving cavity; and
the plurality of first bearings being distributed about a centerline of the first main body along a staggered pattern.

10. The rotary tattoo machine with improved dampening as claimed in claim 1 comprises:
the dampening slider further comprises a second bearing-receiving cavity;
the second bearing-receiving cavity traversing into the second main body from a rear surface of the second main body;
the plurality of second bearings being positioned within the second bearing-receiving cavity; and
the plurality of second bearings being distributed about a centerline of the second main body along a staggered pattern.

11. A rotary tattoo machine with improved dampening comprises:
a tattoo machine housing;
a second spring;
a reciprocating slider comprises a first main body, a plurality of first bearings, a second channel, and a removable endcap;
a dampening slider comprises a second main body, a plurality of second bearings, a needle-connecting rod, and a first spring;
a first channel traversing into the tattoo machine housing;
each of the plurality of first bearings being rotatably connected along the first main body;
the plurality of first bearings being slidably engaged with the first channel;
each of the plurality of second bearings being rotatably connected along the second main body;
the second channel traversing into the first main body, oriented parallel to the first channel;
the second channel and the plurality of first bearings being oppositely positioned of each other across the first main body;
the plurality of second bearings being slidably engaged with the second channel;
the needle-connecting rod being perpendicularly and adjacently connected to the second main body, opposite the plurality of second bearings;
the removable endcap being attached to the first main body, adjacent to an inlet of the second channel;
the second spring being oriented parallel to the first channel;
the second spring being externally connected to the first main body, opposite the removable endcap; and
the second spring being positioned within the first channel.

12. The rotary tattoo machine with improved dampening as claimed in claim 11 comprises:
a dampening adjustment assembly;
the dampening adjustment assembly comprises a thumbscrew, a first nut, and a second nut;
the thumbscrew being positioned collinear with second channel;
the first nut being positioned adjacent to the removable endcap, opposite the second channel;
the second nut being positioned within the second channel, parallel to the first nut;
the thumbscrew traversing through the first nut, the removable endcap, and the second nut;
the first spring being positioned within the second channel, concentric with the thumbscrew; and
the first spring being positioned in between the first nut and the second main body.

13. The rotary tattoo machine with improved dampening as claimed in claim 11 comprises:
the tattoo machine housing comprises a motor-receiving tube and a tower housing;
the tower housing being adjacently and perpendicularly connected to the motor-receiving tube;
the first channel traversing into the tower housing from a top surface of the tower housing; and
the first channel intersecting a front surface of the tower housing.

14. The rotary tattoo machine with improved dampening as claimed in claim 11 comprises:
a motor;
a rotary-to-linear mechanical linkage;
the motor being mounted within a motor-receiving tube of the tattoo machine housing;
an output shaft of the motor being positioned adjacent to the first channel; and
the output shaft being mechanically linked to the first main body by the rotary-to-linear mechanical linkage.

15. The rotary tattoo machine with improved dampening as claimed in claim 11 comprises:
a rotary-to-linear mechanical linkage;
a motor;
the rotary-to-linear mechanical linkage comprises a connecting arm and an interconnecting disk;
the interconnecting disk being concentrically connected to an output shaft of the motor;
a first end of the connecting arm being pivotably connected to the interconnecting disk;
the first end being positioned radially offset from a main axis of the output shaft;
a second end of the connecting arm being adjacently positioned to the first main body, opposite the plurality of first bearings; and
the second end being pivotably connected to the first main body.

16. The rotary tattoo machine with improved dampening as claimed in claim 11 comprises:
a rotary-to-linear mechanical linkage;
a motor;
the rotary-to-linear mechanical linkage comprises a cam, a translating arm, and a cam-receiving cavity;
the cam being axially connected to an output shaft of the motor;
the translating arm being adjacently connected to the first main body, opposite the removable endcap;
the cam-receiving cavity traversing into the translating arm; and
the cam being perimetrically engaged within the cam-receiving cavity.

17. The rotary tattoo machine with improved dampening as claimed in claim 11 comprises:
a needle bar;
the needle bar being positioned adjacent to the needle-connecting rod;
the needle bar being oriented parallel to the first channel; and
the needle bar being perpendicularly attached to the needle-connecting rod.

18. The rotary tattoo machine with improved dampening as claimed in claim 11 comprises:
the reciprocating slider further comprises a first bearing-receiving cavity;
the first bearing-receiving cavity traversing into the first main body from a rear surface of the first main body;
the plurality of first bearings being positioned within the first bearing-receiving cavity; and
the plurality of first bearings being distributed about a centerline of the first main body along a staggered pattern.

19. The rotary tattoo machine with improved dampening as claimed in claim 11 comprises:
the dampening slider further comprises a second bearing-receiving cavity;
the second bearing-receiving cavity traversing into the second main body from a rear surface of the second main body;
the plurality of second bearings being positioned within the second bearing-receiving cavity; and
the plurality of second bearings being distributed about a centerline of the second main body along a staggered pattern.
